(19) United States
(12) Patent Application Publication
(10) Pub. No.: US 2020/0230172 A1
(43) Pub. Date: Jul. 23, 2020

(54) STEM CELL CONDITIONED MEDIA FOR CLINICAL AND COSMETIC APPLICATIONS

(71) Applicant: OCT THERAPIES AND RESEARCH PRIVATE LIMITED, Mulund (West) (IN)

(72) Inventor: Caroline Evette MATHEN, Mumbai, Maharashtra (IN)

(21) Appl. No.: 16/486,506

(22) PCT Filed: Feb. 16, 2018
(86) PCT No.: PCT/IN2018/050078

§ 371 Date: Aug. 16, 2019

(30) Foreign Application Priority Data
Feb. 16, 2017 (IN) 201721005531

(51) Int. Cl. A61K 35/12; A61K 8/98; C12N 5/0775; A61K 47/36; A61K 47/10; A61K 47/12; A61K 47/18; A61P 17/14; A61K 8/34; A61K 8/368; A61K 8/73; A61K 8/44; A61Q 7/00

(52) U.S. Cl. CPC A61K 35/12; A61K 2800/524; C12N 5/0662; A61K 47/36; A61K 47/10; A61K 47/12; A61K 47/183; A61P 17/14; A61K 8/345; A61K 8/368; A61K 8/73; A61K 8/44; A61Q 7/00; A61K 2800/51; A61K 8/981

(57) ABSTRACT

The present invention relates to a cell-free, stem cell conditioned medium and a process for preparation thereof. Further, the present invention relates to a therapeutic composition comprising the said stem cell conditioned medium for therapeutic and cosmetic purposes. Additionally, the present invention relates to a method for treating dermatological conditions and aiding in hair regeneration by administering the composition of the present invention.

### TECHNICAL FIELD OF THE INVENTION

The present invention relates to a cell-free conditioned medium comprising beneficial stem cell factors secreted by human stem cells and a process for preparation thereof.

Further, the present invention relates to a therapeutic composition comprising a cell-free culture medium conditioned by stem cells or a fraction thereof for therapeutic and cosmetic purposes.

Additionally, the present invention relates to a method for treating dermatological conditions and aiding in hair regeneration by administering the composition of the present invention.

### BACKGROUND AND PRIOR ART OF THE INVENTION

Numerous dermatological conditions, wound healing, age-related skin disorders, psoriasis, eczema, dermatitis, acne, skin irritation, skin rash and dry skin, and other dermatological diseases are common skin ailments and there is an obvious need to treat them effectively. Additionally, there is an increasing demand for people to look their best by reversing skin damage or hair loss. Both conditions may be due to intrinsic factors such as Androgenetic Alopecia (AGA) and Telogen effluvium and extrinsic factors including stress, pollution and toxins. Wound injuries in surgery, accidents, ulcers or burn related traumas are other examples in which there is a need for accelerated wound healing.

Premature baldness and age related hair loss is a common problem with male and female pattern baldness. Many researchers have aimed at resolving hair and dermatological associated problems using various approaches. Medicines today are drug driven, overruled by antibiotics, chemotherapy and other pharmaceutical products. The most common approach with respect to hair regrowth is the application of FDA approved drugs Minoxidil and/or Finasteride, or hair transplant surgery. Although generally well tolerated, these drugs have several well documented side effects viz., allergic reactions, dizziness, diarrhea and nausea and are contra-indicated in certain medical conditions.

Additionally, use of Minoxidil is a lifetime commitment as discontinuing usage regresses hair growth to the original condition. The surgical approach to hair loss is hair transplantation which is invasive and depends on the surgeon's skill. Multiple sessions may be necessary to affect an aesthetic look. Since this is a surgical procedure it entails absence from work and risks associated with surgeries. For skin rejuvenation, many approaches are well known including use of animal derived collagen, chemical peels, fillers and Botox, however they may cause allergic reactions, facial paralysis or other side effects.

In light of the drawbacks of products meant for treatment of skin and hair conditions, there is a need to have a biocompatible hair and skin care product, thus potentially circumventing adverse health reactions. This would ensure gentle and beneficial skin and hair care which could possibly reverse damage. The medicine of the future will be based on cell based or cell-free therapies, focused on repair and regeneration or reactivation of tissues and organs. Thus, diseased cells can be replaced by healthy differentiated stem cells or alternately can be persuaded to become healthy by paracrine interactions of stem cells.

Stem cell-derived conditioned medium has been addressed to be a promising prospect for regenerative medicine. A review article by J. Pawitan et al published in BioMed Research International, Volume 2014, Article ID 965849, analyses results of different stem cell-derived conditioned media on various diseases. However, standardized methods of production and validation of components of a conditioned medium for therapeutic purposes and for administration in human beings need to be conducted.

Growth factors (GFs) are essentially proteins that synchronize cell growth, differentiation and proliferation under controlled growth environment. Synergistic interactions of multiple growth factors in human scalp or skin control and regulate hair and skin regeneration respectively. GFs have been proven to impact diverse mechanisms of action in skin repair and rejuvenation with many GFs working in a synchronized manner either independently or synergistically. Intrinsic and extrinsic factors of the scalp and skin reduce levels of endogenous GFs as well as the number and functionality of dermal papilla cells or hair follicle progenitors. Supplementing the scalp or skin's endogenous GFs may enhance natural repair processes and help reverse damage caused by intrinsic and extrinsic factors. Thus a tiny proportion of topically applied GFs penetrating into the dermis can elicit a cell-mediated response leading to desirable and aesthetic outcomes. A synergistic blend of antioxidants, vitamins, amino acids and conditioners with physiologically balanced GFs provides an original and broad paradigm of hair regeneration as well as skin rejuvenation in aesthetic modalities.

R. Moghadasali et al (Experimental and Toxicologic Pathology 65 (2013) 595-600) have investigated the role of human Mesenchymal stem cells (MSC)-derived conditioned medium in inhibition of nephrotoxicity by renal proximal tubular cells. They suggest identification of a specific set of cytokines and growth factors secreted by the MSC's that are implicated in the protective mechanism on these cells leading to renal repair in-vivo to provide a novel therapy.

Apart from stem cells, conditioned media derived from the culturing process has tremendous potential for therapeutic and cosmetic applications as mentioned in the featured prior arts. A similar rationale that stem cells used for therapies should avoid animal derived products would also apply to conditioned media derived from stem cell culture. Thus, there is an urgent need, to manufacture a human serum based medium to culture stem cells economically, which is xeno-free and therefore safe from ethical and regulatory perspectives. Hence, the inventor of the present invention has resolved issues of animal based components interfering in cell based or cell-free products.

Therefore, in an attempt to provide a convenient, economically feasible and non-invasive composition having no side-effects to treat dermatological conditions and to aid in hair regeneration, the present inventors have made available a cell-free therapeutic and cosmetic composition comprising a stem cell conditioned medium supplemented with human serum.

### SUMMARY OF THE INVENTION

In the most preferred aspect, the present invention provides a process for preparing stem cell conditioned medium (CM) for clinical and cosmetic applications comprising;
(i) supplementing 5% to 30% serum extracted from fresh frozen plasma (FFP) or cryo-depleted plasma (CDP) in a culture medium;
(ii) cultivating stem cells in the said culture medium of step (i) for a period of 24 hours to 72 hours to allow the secretion of metabolites selected from the group comprising exosomes, micro-vesicles, soluble proteins, cytokines, chemokines, enzymes, hormones, regulatory and anti-inflammatory factors, signaling molecules and growth factors to obtain stem cell conditioned medium;
(iii) harvesting the conditioned culture medium to separate whole cells and cellular debris to obtain a cell free stem cell conditioned medium; and
(iv) aliquoting the stem cell free conditioned medium under sterile conditions;
   wherein the said conditioned medium comprises at least two metabolites in a concentration ranging from about 50 pg/ml to about 3000 pg/ml.

The process for extracting serum employed for supplementing the culture medium in step (i) of the process for preparing stem cell conditioned medium meant for cultivating mesenchymal stem cells comprises;
a) preparing the growth supplement of human serum by pooling fresh frozen plasma (FFP) or cryo-depleted plasma (CDP) from between single to multiple lots under sterile conditions to reduce lot to lot variability of the biological component.
b) treating FFP or CDP with 2%-20% sterile calcium chloride (CaCl₂)) (0.025M-1M), to separate human serum and to remove clotting factors and cryoprecipitate present in plasma; followed by allowing the clotting process to proceed at room temperature for 2-8 hours and then at 4-8° C. overnight to obtain serum from the clot;
c) separating calcium chloride treated FFP or CDP under sterile conditions followed by inactivating the complement system by maintaining serum in a water bath at 56° C. for 30 mins followed by cooling;
d) adding 0.01%-5% peracetic acid (PAA) to serum obtained in step (c) to oxidize and inactivate viruses or bacteria present and keeping the same for 30 minutes to 1 hour;
e) adding sterile sodium bisulphite at a concentration of between 100-200 mg/100 ml to step (d), to neutralize the effect of PAA;
f) filtering the serum of step (e) first through 0.8 μm followed by filtration through 0.2 μm filter, and aliquoting in sterile containers followed by storing at −20° C.

Accordingly, the stem cell conditioned medium comprises human Fibroblast growth factor (hFGF), human Granulocyte Colony Stimulating factor (hGCSF), human Hepatocyte growth factor (hHGF), Interleukin 1 receptor agonist (IL-1ra), human vascular endothelial growth factor (hVEGF) and Interleukin-6 (IL-6).

In yet another aspect, the present invention provides a method for treating dermatological or skin ailments, comprising topically applying the composition comprising stem cell conditioned medium onto a portion of affected human skin to encourage healthy growth or healing.

In one more aspect, the present invention provides a method of aiding in hair regeneration, comprising topically applying the composition containing stem cell conditioned medium onto a portion of affected scalp to encourage healthy growth.

In a further aspect, the present invention provides a cosmetic or therapeutic composition comprising the stem cell conditioned medium for use in treatment of dermatological conditions and in aiding hair regeneration.

### BRIEF DESCRIPTION OF THE DRAWINGS

FIG. 1 illustrates the Interleukin-10 (IL-10) expression in treatment groups;

FIG. 2 illustrates the Interleukin-6 (IL-6) expression in treatment groups;

FIG. 3 depicts the rate of wound healing measurements revealing that there was significant difference with respect to the time points (p<0.05****) as well as the different groups (p<0.05*);

FIG. 4 depicts concentration of growth factors in the cell free stem cell CM.

FIG. 5 depicts the hair pattern growth in subject suffering from balding caused due to Androgenetic alopecia (AGA), telogen effluvium or female pattern hair loss.

Biological material used: Primary cultures of Human Mesenchymal stem cells are derived from human umbilical cord matrix or Wharton's Jelly. The human umbilical cords are collected from normal or elective caesarian deliveries. Embryonic stem cells can be generated from discarded embryos and cultured using the above method. Induced Pluripotent stem cells can be cultured for personalized regenerative medicine (autologous) and cultured using the above method.

### DETAILED DESCRIPTION OF THE INVENTION

The term “adult stem cells” used herein denotes human mesenchymal stem cells (MSCs) from bone marrow, adipose tissue, umbilical cord blood or umbilical cord matrix, pericytes, endothelial progenitor cells, hematopoietic stem cells, monocytes, macrophages, keratinocytes, fibroblasts, and any other cell type, either alone or in a combination that generate or secrete proteins, growth factors, chemokines, cytokines or regulatory mediators.

The said “adult stem cells” are obtained from a single donor, or maybe pooled from multiple donors. The said cells may be used immediately upon donation or may be used only after being cryopreserved for a period of time.

The term “beneficial stem cell factors” used herein refers to factors that may be secreted from adult stem cells. The term denotes factors that have a desirable or positive effect on all cells of the human body due to paracrine interactions. Beneficial factors are adult stem cell secretory molecules which augment the culture medium so that it becomes conditioned by the stem cells.

The “stem cell factors” are selected from but not limited to exosomes, micro-vesicles, growth factors, regulatory factors, hormones, enzymes, cytokines, chemokines, lymphokines and peptides or combinations thereof.

MSCs, pericytes and endothelial progenitor cells or their mixed populations may be obtained from different tissues in the human body such as bone marrow, fat or adipose tissue, umbilical cord blood, Wharton's Jelly, placental membranes or amniotic fluid or any other suitable source and from normal or elective caesarian deliveries. Stem cells and progenitor cells may also be sourced from peripheral blood; mobilized or activated peripheral blood, cord blood, menstrual blood or fat tissue, or any tissue in the body that may be an efficient source for stem, progenitor or regenerative cells.

The adult stem cells may also be obtained from an entire umbilical cord without removal of blood vessels or any other tissue and mixed cell populations maybe obtained comprising MSCs, endothelial progenitor cells, fibroblasts and pericytes.

The invention will now be described in detail in connection with certain preferred and optional embodiments, so that various aspects thereof may be more fully understood and appreciated.

In the most preferred embodiment, the present invention provides a process for preparing a stem cell conditioned medium for clinical and cosmetic applications comprising;
(i) supplementing 5% to 30% serum extracted from fresh frozen plasma (FFP) or cryo-depleted plasma (CDP) in a culture medium;
(ii) cultivating stem cells in the said culture medium of step (i) for a period of 24 hours to 72 hours to allow secretion of metabolites selected from the group comprising exosomes, micro-vesicles, soluble proteins, cytokines, chemokines, enzymes, hormones, regulatory and anti-inflammatory factors, signaling molecules and growth factors to obtain stem cell conditioned medium;
(iii) harvesting the conditioned culture medium to separate whole cells and cellular debris to obtain a cell free stem cell conditioned medium; and
(iv) aliquoting the stem cell free conditioned medium under sterile conditions;
   wherein the said conditioned medium comprises at least two metabolites in a concentration ranging from about 50 pg/ml to about 3000 pg/ml.

The process for extracting serum from fresh frozen plasma (FFP) or cryo-depleted plasma (CDP) has been disclosed by the present inventor in an earlier Indian Patent Application No. 1471/MUM/2014. However, the process for preparation of a stem cell conditioned medium by employing the serum in specific concentrations so as to obtain the secretion of growth factors in specific concentrations is not disclosed.

The process for extracting serum employed for supplementing the culture medium in step (i) of the process for preparing stem cell conditioned medium meant for cultivating mesenchymal stem cells;
a) preparing the growth supplement of human serum by pooling fresh frozen plasma (FFP) or cryo-depleted plasma (CDP) from between single to multiple lots under sterile conditions to reduce lot to lot variability of the biological component.
b) treating FFP or CDP with 2%-20% sterile calcium chloride (CaCl₂)) (0.025M-1M), to separate human serum and to remove clotting factors and cryoprecipitate present in plasma; followed by allowing the clotting process to proceed at room temperature for 2-8 hours and then at 4-8° C. overnight to obtain serum from the clot;
c) separating calcium chloride treated FFP or CDP under sterile conditions followed by inactivating the complement system by maintaining serum in a water bath at 56° C. for 30 mins followed by cooling;
d) adding 0.01%-5% peracetic acid (PAA) to serum obtained in step (c) to oxidize and inactivate viruses or bacteria present and keeping the same for 30 minutes to 1 hour;
e) adding sterile sodium bisulphite at a concentration of between 100-200 mg/100 ml to step (d), to neutralize the effect of PAA;
f) filtering the serum of step (e) first through 0.8 μm followed by filtration through 0.2 μm filter, and aliquoting in sterile containers followed by storing at −20° C.

In another preferred embodiment, the present invention provides the stem cells cultivated in the culture medium is selected from the group comprising mesenchymal stem cells (MSCs), embryonic stem cells and induced pluripotent stem cells (iPSCs) and/or stem cells derived from pericytes, endothelial progenitor cells, hematopoietic stem cells, progenitor cells, monocytes, macrophages, keratinocytes or fibroblasts.

The present invention provides culturing stem cells in a culture medium, comprising processing the umbilical cord and culturing and cryo-preserving stem cells being completely xeno-free. MSCs used for culturing may also be derived from bone marrow, umbilical cord blood, placental tissues, adipose tissue and amniotic tissues or any other suitable source.

In an embodiment, the present invention provides cultivation of stem cells in a culture medium supplemented with 5% to 30% serum including but not limited to Dulbecco's modified essential medium (DMEM), alpha MEM, Ham's F12, DMEM/F12, Minimum Essential Medium (MEM) and keratinocyte medium.

The human serum recovered from human fresh frozen plasma (FFP) or cryo depleted plasma (CDP) can be used as a growth supplement in a culture media including but not limited to Minimum Essential Medium (MEM), Ham's F12, DMEM/F12 or Minimum Essential Medium (MEM) for culture and cryopreservation of various cell types. For dermatological applications, stem cells are cultured in keratinocyte medium.

The culture medium is devised so as to support the growth and maintenance of stem cells; allow secretion of exosomes, micro-vesicles, growth factors, cytokines, chemokines and soluble beneficial factors from the stem cells into the medium so as to form a stem cell conditioned medium.

In a further embodiment, stem cells in serum supplemented medium are cultured in step (ii) at a temperature ranging from 36°-37° C. and at 4%-20% CO₂ and at a density of between 3000 to 60000 cells/cm² for a duration of 24 hrs to 72 hrs.

In one preferred embodiment, the present invention provides a stem cell conditioned medium comprising human Fibroblast growth factor (hFGF) ranging from about 100 to about 1500 pg/ml, human Granulocyte Colony Stimulating factor (hGCSF) can be between 50-1500 pg/ml, human Hepatocyte growth factor (hHGF) can be between 50-3000 pg/ml, Interleukin 1 receptor agonist (IL-1ra) can be between 100-1500 pg/ml, human vascular endothelial growth factor (hVEGF) can be between 10-1000 pg/ml and Interleukin-6 (IL-6) ranging from about 100 to about 2000 pg/ml. FIG. 4 depicts concentrations of each of the metabolites/growth factors secreted in culture medium supplemented with serum by stem cells.

At least two of these growth factors should be present for the desired efficacy along with an in vitro anti-oxidant activity ranging from 25% to 90%. This anti-oxidant activity of the stem cell conditioned medium is estimated using 2,2-diphenyl-1-picryl hydrazyl (DPPH) assay.

In yet another preferred embodiment, the present invention provides a stem cell conditioned medium comprising growth factors selected from the above group consisting of exosomes, micro-vesicles, soluble proteins, cytokines, chemokines, enzymes, hormones, regulatory and anti-inflammatory factors, signaling molecules and growth factors wherein the said conditioned medium comprising at least two metabolites in a concentration ranging from about 50 pg/ml to about 3000 pg/ml.

The conditioned medium is prepared by culturing MSCs in a suitable culture medium for around twenty four hours to approximately three days so as to allow cell secretions to leach into culture medium, to form a physiologically balanced composition of beneficial cytokines, growth factors and proteins.

The resultant stem cell conditioned medium obtained by the present process is a pale yellow aqueous solution with a specific gravity of between 0.99 to 1.2.

In another embodiment, the present inventors have provided the stability of the stem cell conditioned medium for a period up to 2 years when stored at temperatures as less as −20° C. The stability is tested by total protein content, anti-oxidant activity and presence of growth factors.

After harvesting the conditioned medium, the supernatant that is free from cellular matter or debris is subjected to filtering to ensure sterility of the conditioned medium. An aliquot of the conditioned medium obtained from aforesaid method is subjected to sterility testing for bacteria and fungus using Thioglycollate and Saboraud Dextrose Broth (SB) media respectively. The aliquots are also tested for presence of mycoplasma and endotoxin.

In another preferred embodiment, the present invention provides an additional step comprising of filtering or centrifugation to remove whole cells or debris from the conditioned medium in order to obtain a cell-free extract. The process includes harvesting the extract such that removing the extract involves removing the cell culture supernatant resulting in a cell-free extract from cultured cells comprising the harvested conditioned medium. Whole cells and cellular debris are excluded.

In yet another embodiment, the present invention provides removal of supernatant conditioned medium from cumulative passages from single or multiple lots. The extract or supernatant from the culture medium may be from early passages of the adult stem cells or from late passages or cell lines derived from them.

The extract or the supernatant may be taken from the primary passage or from a later passage and either may be used in the skin or hair care formulation. It could also be a cumulative collection of various passages across different cell lots obtained from multiple donors. A preferable approach could be by preparing mixed master cell bank consisting of early passages from minimum 2 to multiple lots across different donors to get uniform end products. Furthermore, incorporating extracts from the conditioned medium into cosmetic or pharmaceutical formulations for skin care, hair care, wound healing or other therapeutic indications, the extract may be used after filtering or centrifugation to remove the cell debris so that the final extract is a cell free product. This extract maybe used in whole, concentrated or fractionated to isolate specific components which may be deemed to be desirable as per the intended application.

The conditions of the present processes maybe modified or optimized by altering Oxygen concentrations or the type of culture for e.g. 3D cultures or hollow fiber bioreactors to alter the secretions of the adult stem cells into the culture media.

The conditioned media maybe further concentrated by methods known to those in the art, for e.g. by centrifugation, protein separation, lyophilization and so on. This customized conditioned media can be formulated into formulations for cosmetic and therapeutic applications.

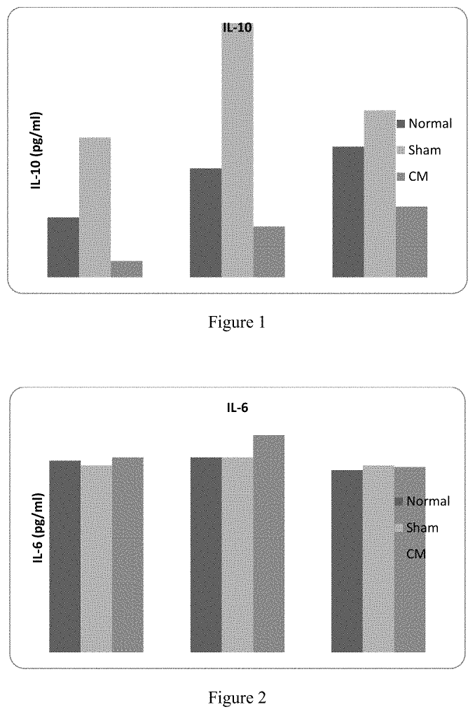

The conditioned media obtained by the present process is tested by in vitro and in vivo methods for safety and efficacy. The efficacy of the stem cell conditioned medium formulated in a therapeutic composition for the treatment of wound healing in rat showed reduced expression of Interleukin-10 (IL10) due to lower levels of inflammation and infection. FIG. 1 describes lower concentrations of the IL-10 cytokine across the treatment group administered with the present composition comprising the stem cell conditioned medium. Employing the conditioned medium of the present invention, it is observed that the area of wound is reduced significantly 24 days after sub-cutaneous injections applying the formulation comprising the conditioned medium.

The safety of human Mesenchymal stem cell conditioned medium is indicated by the non-elicitation of any adverse immune reaction when injected or topically applied on wounds. Moreover, the application of the composition obtained by the present process does not elicit any adverse reaction in other mammalian species.

### Therapeutic Composition:

In one more preferred embodiment, the present invention provides a therapeutic composition for hair regeneration and cosmetic applications comprising;

a. a stem cell condition medium in an amount ranging from about 0.5% to 100%; and

b. one or more pharmaceutically acceptable excipients;

wherein the said medium is obtained by cultivating stem cells in a culture medium supplemented with 5% to 30% serum extracted from fresh frozen plasma (FFP) or cryo-depleted plasma (CDP).

The present invention provides a therapeutic composition comprising a conditioned medium containing beneficial factors selected from the group consisting of exosomes, micro-vesicles, soluble proteins, cytokines, chemokines, enzymes, hormones, regulatory and anti-inflammatory factors, signaling molecules and growth factors.

In another preferred embodiment, the present invention provides a therapeutic composition comprising growth factors/metabolites in a concentration ranging from about 50 pg/ml to about 3000 pg/ml. The growth factors/metabolites may include but not limited to the group consisting of human fibroblast growth factors (hFGF), human hepatocyte growth factors (hHGF), human vascular endothelial growth factors (hVEGF), human Granulocyte Colony Stimulating factor (hGCSF), Interleukin 1 receptor agonist (IL-1ra) and Interleukin-6 (IL-6). Cytokines comprised in the therapeutic composition of the present invention are selected from the group consisting of G-CSF, interleukins and interferons. Protein preferably present is human serum albumin.

More specifically, the stem cell conditioned medium comprises human Fibroblast growth factor (hFGF) in a concentration ranging from 100 to 1500 pg/ml, human Granulocyte Colony Stimulating factor (hGCSF) in a concentration ranging from 50 to 1500 pg/ml, human Hepatocyte growth factor (hHGF) in a concentration ranging from 50 to 3000 pg/ml, Interleukin 1 receptor agonist (IL-1ra) in a concentration ranging from 100 to 1500 pg/ml, human vascular endothelial growth factor (hVEGF) in a concentration ranging from 10 to 1000 pg/ml and Interleukin-6 (IL-6) in a concentration ranging from 100 to 2000 pg/ml.

The present therapeutic and cosmetic composition is formulated as an aqueous formulation, gel, lyophilized preparation, ointments, gels, creams, serums, mask, shampoos, lotions, and intravenous, sub-cutaneous or parenteral formulations. The said topical formulations such as ointments, gels, creams, serums, shampoos, lotions are formulated such that they remain on skin or scalp and involve daily applications. The formulated composition is stable for a period of up to 3 years.

The pharmaceutically acceptable excipient is selected from the group comprising emulsifying agents, antioxidants, buffering agents, solubilizers and solvents. Accordingly, the emulsifying agent used is xanthan gum. The antioxidant is selected from the group consisting of edetate disodium, sodium sulphite, sodium metabisulfite, propyl gallate, edetate trisodium, tocopherol derivatives, butylated hydroxyl toluene, butylated hydroxyl anisole, ascorbic acid, fumaric acid, malic acid, and citric acid. The buffering agents are selected from the group comprising sodium hydroxide, potassium hydroxide, ammonia, hydrochloric acid, acetic acid, lactic acid and citric acid. The solvent/solubilizer is selected from the group consisting of propylene glycol, polyethylene glycol, ethylene glycol, butylene glycol, and hexylene glycol.

In yet another preferred embodiment, the present invention provides the pharmaceutically acceptable excipients selected from the group consisting of;
(i) an emulsifying agent as xanthan gum in a concentration ranging from 0.01-10% by weight of the composition,
(ii) a humectant as Glycerin between 0.5-10% by weight,
(iii) a solubilizer as propylene glycol in a concentration ranging from 0.05-10% by weight,
(iv) an anti-microbial preservatives selected from as sodium benzoate and potassium sorbate in a concentration ranging from 0.03-5% by weight, and
(v) an anti-oxidant selected from EDTA in a concentration ranging from 0.05%-2% by weight.

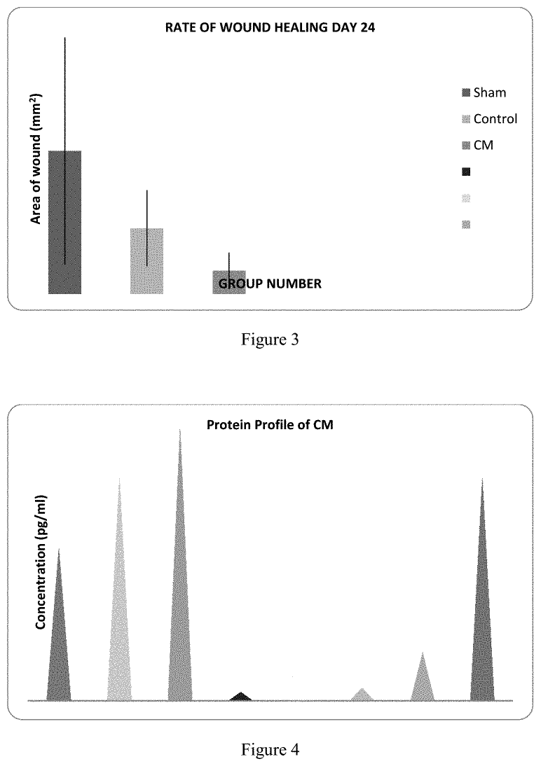

Accordingly, a topical formulation comprising the conditioned culture medium can be administered as an ointment, skin gel, cream, hair or skin serum, shampoo, conditioner and lotions to lend a conditioning or improving property which positively impacts appearance of skin or hair in a non-invasive manner facilitating ease of convenience. Alternately the formulation maybe administered as sub-cutaneous, dermally, parenterally or suitable mode of delivery for therapeutic or cosmetic applications. The conditioned media can be incorporated into dressings such as gauze, band-aids, collagen, hydrogels or suitable scaffolds for wound healing or other cosmetic purposes. Substantial reduction in area of wound on 24th day of treatment with the present therapeutic composition (FIG. 3) is observed. Alternately, conditioned media maybe incorporated into a beauty face pack for skin rejuvenating effect or into hair mask for scalp conditioning and hair regeneration.

The topical formulation is prepared so as to maintain stability of the stem cell derived factors and also its ability to permeate through skin and not degrade in skin. Moreover, it is formulated so that it achieves desired release rates.

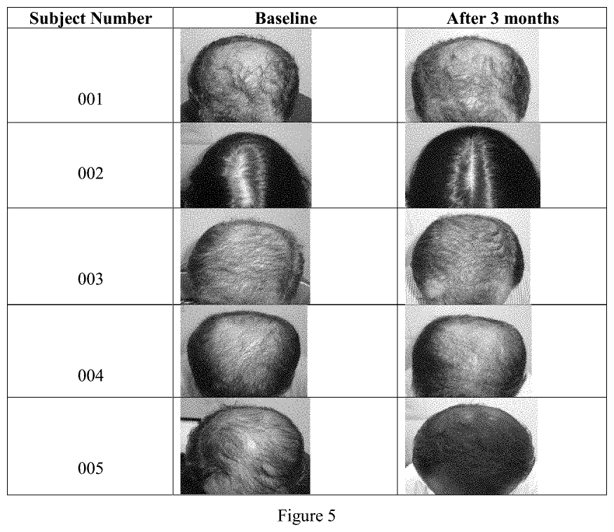

Topical formulations for purposes of regeneration of hair involve cell-free extracts in a shampoo, hair serum, hair mask or conditioner. Accordingly, the said composition can be formulated into a shampoo with one or more pharmaceutically acceptable excipients. FIG. 5 describes significant increase in hair growth over a period of 3 months compared to base line observations.

The present formulation can be administered either topically or by infusions to treat diseased tissues, in particular application to the skin or scalp either directly in combination with derma roller, electroporation or energy based or other suitable devices to enable dermal delivery or in a suitable formulation would help support cell turnover. This would aid in skin rejuvenation, in reversal of skin damage and in hair regeneration. The cell free conditioned medium may also be used for wound healing in acute and chronic wounds, either directly or formulated into an appropriate formulation.

Use: The present therapeutic composition may be employed in treatment of numerous dermatological conditions, selected from but not limited to wounds, age-related skin disorders, psoriasis, eczema, dermatitis, acne, skin irritation, skin rash and dry skin. Wound injuries in surgery, accidents, ulcers or burn related traumas are other examples in which there is a need for accelerated wound healing. Additionally, the present composition can be employed for reversing hair loss caused due to intrinsic and extrinsic processes.

### Method of Treatment: Delivery Route

The application method may also include a step of formulating the extract into a cosmetic or pharmaceutical formulation to facilitate ease of convenience to apply or to increase the stability or for other reasons, and then applying the complete formulation to the skin or scalp.

In yet another embodiment, the extract may be used in part or whole, as a parenteral or infusion administration to either treat a particular condition such as wound healing, stabilization of burn victims or as an adjuvant for Graft versus host disease (GVHD) or donor transplant cases or for patients recovering from radiation treatment or for anti-inflammatory applications.

In one more embodiment, the present invention provides a method of improving the quality and health of human hair and scalp. This method may further include a step of topical application to human scalp of a stem cell-free extract of a conditioned medium and/or components of cell-free extract. The step of topical application to human hair may involve massaging onto hair either at scalp or onto hair directly.

In one embodiment, the present invention provides a method of treating dermatological or skin ailments, comprising topically applying the composition comprising stem cell conditioned medium onto a portion of affected human skin to encourage healthy growth or healing. The extract maybe directly applied onto the skin, or combined with a dermaroller, electroporation device, energy based or other suitable devices to allow beneficial factors to penetrate and enter the dermis.

In one more embodiment, the present invention provides a method of aiding in the regeneration of hair, comprising topically applying the composition comprising stem cell conditioned medium onto a portion of affected scalp to encourage healthy growth. The extract maybe directly applied on the scalp, or combined with a procedure of using dermaroller, electroporation, energy based or other suitable devices in order to allow the beneficial factors to penetrate and enter the dermis.

Further, the therapeutic composition can be employed as an adjuvant for Graft versus host disease (GVHD), donor transplant cases or for patients recovering from radiation treatment or for anti-inflammatory applications. Other indications treated include cardiac, renal, hepatic, pancreatic, graft versus host disease, neurodegenerative disorders, spinal cord ailments and any other new indication.

### EXAMPLES

Following examples are given by way of illustration therefore should not be construed to limit the scope of the invention.

### Examples

### Example 1: Blood Collection

The Human plasma or cryo-depleted plasma (AB positive) was collected in a closed system by certified blood banks. Blood bags which tested negative for infectious diseases including but not limited to HIV 1 and 2, HbsAg, HCV (Hepatitis), syphilis (VDRL) and malarial parasites were further transported cold to the laboratory and processed under sterile conditions using aseptic techniques, to recover serum such that it is suitable to support culture of clinical grade quality human Mesenchymal cells (MSCs) which can be used for clinical applications. The serum from pooled AB positive plasma or cryo-depleted plasma (CDP) was recovered and further processed to neutralize viruses, bacteria and mycoplasma as per the method specified by Sprössig, 1976; and Wutzler, 1975. The pooling was from between 5 to multiple donor lots as per the processing capacity and was done to reduce lot to lot variability of a biological component.

### Example 2

Process for preparation of stem cell conditioned medium with varying serum concentrations
(a) Human serum recovered in accordance with Example 1 is used to supplement cell culture media such as DMEM, at 10%. The supplemented culture medium is used to culture Mesenchymal stem cells at a cell density ranging from 3000 to 60000 cells/cm² in suitable culture vessels which could be 2-D or 3-D. Cells are incubated at 36° C. and 4 to 20% CO₂ till 70-90% confluent. The stem cell CM is harvested from 24 hours to 3 days, is pooled over cumulative passages and across different lots, filtered, aliquoted and stored at −20° C.
(b) Serum recovered in accordance with Example 1 is used to supplement cell culture medium DMEM/F12 at a 5%. The supplemented culture medium is used to culture Embryonic stem cells at a cell density ranging from 1000 to 30000 cells/cm² in suitable culture vessels The cells are incubated at 37° C. and 5% CO₂ till 70-90% confluency is obtained. The stem cell conditioned media is further subject to treatment as per the process of Example 2(a).
(c) Human serum recovered in accordance with Example 1 is used to supplement cell culture medium DMEM/F12 at a 7%. The supplemented culture medium is used to culture Pluripotent stem cells at a cell density ranging from 1000 to 30000 cells/cm² in suitable culture vessels. The cells are incubated at 37° C. and 5% CO₂ till 70-90% confluency is obtained. The stem cell conditioned media is further subjected to treatment as per the process of Example 2(a).

### Example 3: Experimental Conditions

Total protein content: Total protein content was between 0.5 to 100 mg/ml

Cytokine analysis by ELISA or Multiplexing: The following cytokines were present at the respective concentrations: human Fibroblast growth factor (hFGF) 658 pg/ml, human Granulocyte Colony Stimulating factor (hGCSF) 960 pg/ml, human Hepatocyte growth factor (hHGF) 1175 pg/ml, Interleukin 1 receptor agonist (IL-1ra) 364 pg/ml, human vascular endothelial growth factor (hVEGF) 208 pg/ml and Interleukin-6 (IL-6) 962 pg/ml. At least 2 of these growth factors should be present for the desired efficacy along with an in vitro anti-oxidant activity of between 25-90% using 2,2-diphenyl-1-picryl hydrazyl (DPPH) assay.

Sterility: Negative for aerobic and anerobic bacteria, fungus, mycoplasma and endotoxin pH Between 5.5 to 9.0 pH.

Stable at −20° C. for 2 years, Anti-oxidant activity as estimated by 2,2-diphenyl-1-picrylhydrazyl (DPPH) assay is between 25-90%.

### Example 4: Wound Healing in Rats

In vivo studies were carried out in an animal model of excisional wound healing where MSC derived conditioned media (CM) was used as treatment group in comparison with normal and sham controls. This study was conducted in compliance with all regulatory and ethical approvals to assess wound healing activity of Human MSC derived conditioned media in laboratory Wistar rat. Healthy young adult Wistar rats were allowed to acclimatize and housed to standard experimental laboratory conditions for a period of 9 days before exposure to the present composition. Animals were randomized and allocated in different groups viz. Normal Control, Sham Control and CM. Each group was comprised of 6 rats per group per dose. The wound site was prepared 1 day prior to infliction of excision wound by clipping fur on dorsal thoracic region of the rat. An area of approximately 500 mm² dorsal skin was cut open to create a full skin thickness excision wound. The wound was surgically created under injectable anesthesia by ketamine [100 mg/kg, IM] and xylazine [16 mg/kg, IM]. Treatment was administered by sub-cutaneous injection wherein stem cell CM is present in concentration of 100% and in an amount of 1 ml as a single dose.

Rats were observed for mortality and morbidity twice daily. All visible signs and symptoms such as changes in activity, behavioral pattern, change in skin and fur, eyes and mucous membranes, abnormal growth and other general changes were observed and recorded once daily till the end of the experimental period. Body weights of rat were recorded before initiation of dosing and 7^{th}, 14^{th}, and 24^{th} day of the experimental period. Animals were provided with adequate quantity of feed every day. Measurement of wound closure was recorded on 1^{st}, 4^{th}, 8^{th}, 11^{th} and 24^{th} days of experimental period. Blood was collected on days 4, 11 and 24 for serum cytokine analyses. Animals were humanely sacrificed at the end of the experimental period. Gross pathological examination included an examination of the external surface of the body, all orifices, the cranial, thoracic and abdominal cavities and their contents were observed. Biopsies were collected for histopathology analyses. Raw data was processed and analyzed between controls and the treated groups using statistical software such as Graphpad Prism. The results indicated that no gross pathological changes were observed in the experimental treatment group. Cytokine expression revealed that in the treatment group, IL10 (FIG. 1) was low compared to untreated controls or sham, possibly due to lower levels of inflammatory or infection driven processes and this difference was statistically significant across both time (p<0.05*) and treatment groups (p<0.05***). By day 24, IL10 levels were elevated in the control groups compared to the treatment groups at all-time points with significant difference by the last end point. IL-6 level estimation (FIG. 2) revealed that there was a difference between various time points which was statistically significant (p<0.05**) but not between the groups. Results suggest a lack of immune response by rat immune systems to human MSC derived CM which did not elicit any adverse or immune reaction despite being used in another species. Rate of wound healing measurements in FIG. 3 revealed that there was significant difference with respect to time points (p<0.05****) between sham and CM treated groups. This trend continued over subsequent time points till the final end point and showed faster rate of wound healing in CM treatment group. In histopathology results for PMNL/inflammatory cells, the treatment group had lesser inflammatory cells than the sham. For evaluation of neo-angiogenesis, treatment group had lower values than control or untreated groups. Based on the results, it can be concluded that CM group had promising wound healing potential under experimental conditions used herein.

### Example 5: Hair Regeneration in Humans Employing the Present Composition

A pilot study was carried out to evaluate the safety and efficacy of the Conditioned Media (CM) for hair regeneration on human subjects as a proof of concept experimental study. After obtaining the written informed consent, patients were selected, treated and evaluated for improvement in hair loss. The primary objective of this study was to evaluate efficacy of a hair fall control stem cell based topical product along with nutritional supplements in hairloss patients using Clinical grading and macro photographic evaluation.

The secondary objectives of the research study were as follows:
1. To evaluate improvement in scalp conditions from the baseline.
2. Evaluation of effects of the product in hair texture.
3. Self-assessment of the product by the volunteers in terms of efficacy of treatment and satisfaction.

Number of Subjects:

A total of 15 subjects screened for the study and 15 subjects enrolled in the study and completed 91 days of study. 1 eligible subject did not follow the protocol schedule.

Procedure:

The male and female subjects in the age group of 20-58 years where Men showing Male Pattern Baldness (MPB) grade of II to IV in Hamilton Norwood scale and women showing Female Pattern Hair Loss (FPHL) grade of II to IV on Sinclair scale were enrolled in the study. The primary objective of the experimental therapy was to evaluate the efficacy of a hair fall control stem cell based topical product along with nutritional supplements in hairloss patients using Clinical grading and macro photographic evaluation which were performed on day 0 (baseline), day 35 and day 91. 2 ml of the present therapeutic composition was applied by investigator on the scalp using derma roller at weekly intervals for 3 months and included a total of twelve treatment sessions.

Test Product:

Topical Stem Cell based product was applied using derma roller.

Diagnosis and main criteria for inclusion:

Men showing MPB grade of II to IV in Hamilton Norwood scale and women showing FPHL grade of II to IV on Sinclair scale.

Criteria for Evaluation:

The following parameters were estimated for each of the enrolled study subject.

Efficacy:

The primary outcome measures improvements in hair fall control and hair regeneration, using clinical grading and macro photographic evaluation; and scalp condition at 3 months compared to the respective basal reading.

Safety:

To monitor and record any adverse events reported during the study.

Summary:

This report was analyzed based on 15 subjects' complete data to evaluate the benefits of stem cell based product along with nutritional supplements in the treatment of Hair loss. Age of the evaluated subjects ranged from 20-58 years. Subjects underwent Scalp Examination, MPB grade using Hamilton Norwood or Sinclair scale for females, Macro photographic evaluation, Adverse events and Efficacy parameters.

Safety Results:

No adverse events related to Investigational Product were reported during the treatment period.

Results:

Change in HN/Sinclair scores: 86.6% (FIGS. 5a, 5b and 5c).

Reduction in grades by 1 point in 3 months

Grade IV: 5 patients, Grade III: 2 patients and Grade II: 6 patients

ADRs: None

Patient Feedback:

After 1 month

85% agreed treatment was effective with control of hair fall along with hair growth

78% were highly satisfied with the progress of the treatment

After 3 months

92% agreed treatment was effective, with considerable hair regeneration and reduction in bald spot and 92% were highly satisfied with the treatment

NOTE:

None of the patients complained about any pain, tenderness, redness and no irritation was experienced.

5-10% of young patients showed improvement in control of greying of hair. Self assessment scores improved after application of stem cell based product in terms of efficacy of treatment and satisfaction.

Conclusion:

Stem cell based product caused improvement in hair fall and hair regeneration using clinical grading, macro photographic evaluation and scalp condition.

### Example 6: Formulation of Topical Composition

For the pilot hair study, 2 ml of the filtered stem cell conditioned medium was used directly without any modification of the formulation. The topical formulation is an aqueous, pale yellow solution and was stored at −20° C. It was thawed and used immediately for application in hair regeneration over a three month period. FIGS. 5(a), (b) and (c) depict the increased hair growth over the said time interval.

TABLE 1
Topical formulation
Topical formulation

| Ingredients | | Concentration (% by |
|---|---|---|
| Active | Stem cell conditioned | 0.5-100% |

Excipients

| | | |
|---|---|---|
| Emulsifier | Xanthan gum | 0.01-10% |
| Humectant | Glycerin | 0.5-10% |
| Solubilizer | Propylene glycol | 0.05-10% |
| Preservative | Sodium benzoate | 0.03-5% |
| Preservative | Potassium sorbate | 0.03-5% |
| Anti-oxidant | EDTA | 0.05-2% |

### Example 7: Stability Results

Hair Serum was tested for Freeze thaw and Accelerated stability studies for 7 days and 94 days, respectively. The following observations were noted:

TABLE 2
Freeze Thaw for hair serum
Freeze Thaw for Hair Serum

| | Batch 1 | Batch 2 | Batch 3 |
|---|---|---|---|
| Parameters | Day 7 | Day 7 | Day 7 |
| Colour | T | T | T |
| Texture | S | S | S |
| pH | 5.2 | 5.4 | 5.3 |
| Viscosity | 2700 | 2600 | 2700 |
| Flow | F | F | F |
| Phase separation | NO | NO | NO |
| Precipitation of ingredient | NO | NO | NO |

T—TRANSLUCENT, ST—STABLE, S—SMOOTH, F— FLOWING Viscosity at 25° C., Brookefield Viscometer LV, Spindle 64.6 rpm, in centipoises

TABLE 3
Accelerated Stability Studies
Accelerated Stability Studies for Hair Serum

| Batch 1 | | | | | Batch 2 | | | | Batch 3 | | | |
|---|---|---|---|---|---|---|---|---|---|---|---|---|
| | At | | At | Light | At | | At | Light | At | | At | Light |
| | 4° C. | At RT | 45° C. | Sensitivity | 4° C. | At RT | 45° C. | Sensitivity | 4° C. | At RT | 45° C. | Sensitivity |
| Parameters/ | D 94 | D 94 | D 94 | D 94 | D 94 | D 94 | D 94 | D 94 | D 94 | D 94 | D 94 | D 94 |
| Day(D) | | | | | | | | | | | | |
| Colour | T | T | T | T | T | T | T | T | T | T | T | |
| Texture | S | S | S | S | S | S | S | S | S | S | S | S |
| pH | 5.5 | 4.7 | 5.2 | 5.4 | 5.2 | 5.3 | 5.2 | 5.4 | 5.5 | 5.3 | 5.2 | 5.4 |
| Viscosity | 2700 | 2700 | 2300 | 2400 | 270 | 270 | 2300 | 2400 | 2700 | 2700 | 2300 | 2400 |
| Flow | F | F | F | F | F | F | F | F | F | F | F | F |
| Phase | NO | NO | NO | NO | NO | NO | NO | NO | NO | NO | NO | NO |
| separation | | | | | | | | | | | | |
| Precipitation | NO | NO | NO | NO | NO | NO | NO | NO | NO | NO | NO | NO |
| of ingredient | | | | | | | | | | | | |

T—TRANSLUCENT,
ST—STABLE,
S—SMOOTH,
F—FLOWING Viscosity at 25° C.,
Brookefield Viscometer LV,
Spindle 64.6 rpm, in centipoises

From Table 3, it is evident that the formulated hair serum composition is stable for a period of over 3 months. Similar stability observations were also observed for a period of upto 3 years.

### Advantages of the Invention

Animal derived sources are completely avoided in the present culturing process, leading to a xeno-free composition which is exceptionally safe from the ethical, scientific and regulatory aspects.
Use of a conditioned media helps in mass production of a therapeutic or cosmetic composition for treatment of skin disorders and in hair regeneration.
The use of harmful chemicals is avoided, instead the beneficial cytokines, growth factors and proteins are therapeutic and efficacious at the cellular level, enabling cell-cell cross communication which will gently reverse any signs of cellular damage and restore the required balance in the skin and scalp thereby leading to good skin and hair health.
The present composition facilitates reactivation of hair follicles and also skin rejuvenation using a judicious blend of biocompatible nourishing factors obtained by the present process.

We claim:

1. A process for preparing a stem cell conditioned medium for clinical and cosmetic applications comprising;
(i) supplementing 5% to 30% serum extracted from fresh frozen plasma (FFP) or cryo-depleted plasma (CDP) in a culture medium;
(ii) cultivating stem cells in the said culture medium of step (i) to allow the secretion of metabolites selected from the group comprising exosomes, micro-vesicles, soluble proteins, cytokines, chemokines, enzymes, hormones, regulatory and anti-inflammatory factors, signaling molecules and growth factors to obtain stem cell conditioned medium;
(iii) harvesting the conditioned culture medium to separate whole cells and cellular debris to obtain a cell free stem cell conditioned medium; and
(iv) aliquoting the stem cell free conditioned medium under sterile conditions;
wherein the said conditioned medium comprising at least two metabolites in a concentration ranging from about 50 pg/ml to about 3000 pg/ml.

2. The process for preparing a stem cell conditioned medium according to claim 1, wherein the process for extracting serum from fresh frozen plasma (FFP) or cryo-depleted plasma (CDP) for preparing the supplement comprises;
a) preparing the growth supplement of human serum by pooling fresh frozen plasma (FFP) or cryo-depleted plasma (CDP) from between single to multiple lots to reduce lot to lot variability of the biological component;
b) treating FFP or CDP with 2%-20% calcium chloride to separate human serum and to remove clotting factors and cryoprecipitate in plasma; followed by allowing the clotting process to proceed at room temperature for 2-8 hours and then at 4-8° C. overnight to obtain serum from the clot;
c) separating calcium chloride treated FFP or CDP of step (b) under sterile conditions followed by inactivating the complement system by maintaining serum in a water bath at 56° C. for 30 mins followed by cooling;
d) adding 0.01%-5% peracetic acid (PAA) to serum obtained in step (c) to oxidize and inactivate viruses or bacteria present and keeping the same for 30 minutes to 1 hour;
e) adding sterile sodium bisulphite at a concentration of between 100-200 mg/100 ml to step (d), to neutralize the effect of PAA;
f) filtering the serum of step (e) first through 0.8 μm followed by filtration through 0.2 μm filter, and aliquoting in sterile containers followed by storing at −20° C.

3. The process for preparing a stem cell conditioned medium according to claim 2, wherein the said medium comprises human Fibroblast growth factor (hFGF) in a concentration ranging from 100 to 1500 pg/ml, human Granulocyte Colony Stimulating factor (hGCSF) in a concentration ranging from 50 to 1500 pg/ml, human Hepatocyte growth factor (hHGF) in a concentration ranging from 50 to 3000 pg/ml, Interleukin 1 receptor agonist (IL-1ra) in a concentration ranging from 100 to 1500 pg/ml, human vascular endothelial growth factor (hVEGF) in a concentration ranging from 10 to 1000 pg/ml and Interleukin-6 (IL-6) in a concentration ranging from 100 to 2000 pg/ml.

4. The process for preparing a stem cell conditioned medium according to claim 1, wherein the stem cells cultivated are selected from the group comprising mesenchymal stem cells, embryonic stem cells, induced pluripotent stem cells and/or stem cells derived from pericytes, endothelial progenitor cells, hematopoietic stem cells, progenitor cells, monocytes, macrophages, keratinocytes and fibroblasts.

5. The process for preparing a stem cell conditioned medium according to claim 1, wherein the culture medium is selected from the group comprising Dulbecco's modified essential medium (DMEM), alpha MEM, Ham's F12, DMEM/F12, Minimum Essential Medium (MEM) and keratinocyte medium.

6. A therapeutic composition for hair regeneration and cosmetic applications comprising;
(a) a stem cell condition medium in a concentration ranging from 0.5% to 100% by weight of the composition; and
(b) one or more pharmaceutically acceptable excipients;
wherein the said medium is obtained by cultivating stem cells in a culture medium supplemented with 5% to 30% serum extracted from fresh frozen plasma (FFP) or cryo-depleted plasma (CDP).

7. The therapeutic composition for hair regeneration and cosmetic applications according to claim 6, wherein the said stem cell conditioned medium comprising human Fibroblast growth factor (hFGF) in a concentration ranging from 100 to 1500 pg/ml, human Granulocyte Colony Stimulating factor (hGCSF) in a concentration ranging from 50 to 1500 pg/ml, human Hepatocyte growth factor (hHGF) in a concentration ranging from 50 to 3000 pg/ml, Interleukin 1 receptor agonist (IL-1ra) in a concentration ranging from about 100 to 1500 pg/ml, human vascular endothelial growth factor (hVEGF) in a concentration ranging from about 10 to about 1000 pg/ml and Interleukin-6 (IL-6) in a concentration ranging from about 100 to about 2000 pg/ml.

8. The therapeutic composition according to claim 6, wherein the said composition is formulated as a gel, lyophilized preparation, cream, lotion, ointment, serums, mask, shampoos and lotions.

9. The therapeutic composition according to claim 6, wherein the pharmaceutically acceptable excipient is selected from the group comprising antioxidants, buffering agents, emulsifying agents, solubilizers, and solvents.

10. The therapeutic composition according to claim 9, wherein the pharmaceutically acceptable excipients is selected from the group consisting of;
(i) an emulsifying agent as xanthan gum in a concentration ranging from 0.01-10% by weight of the composition,
(ii) humectant as Glycerin in a concentration ranging from 0.5-10% by weight,
(iii) a solubilizer as propylene glycol in a concentration ranging from 0.05-10% by weight,
(iv) an anti-microbial preservatives selected from as sodium benzoate and potassium sorbate in a concentration ranging from 0.03-5% by weight, and
(v) an anti-oxidant selected from EDTA in a concentration ranging from 0.05%-2% by weight.

11. A method for treating dermatological or skin ailments, comprising topically applying the composition comprising stem cell conditioned medium according to claim 6 onto a portion of affected human skin in an amount of 0.5 mL to 5 mL for a period of 1 month to 3 months.

12. A method of aiding in regeneration of hair, comprising topically applying the composition comprising stem cell conditioned medium according to claim 6 onto a portion of affected scalp in an amount of 0.5 mL to 5 mL for a period of 1 month to 3 months.

13. Use of the composition of claim 6 for treating dermatological or skin ailments and for regeneration of hair.
